(19) United States
(12) Patent Application Publication
(10) Pub. No.: US 2021/0110928 A1
(43) Pub. Date: Apr. 15, 2021

(54) ASSOCIATION OF PROGNOSTIC RADIOMICS PHENOTYPE OF TUMOR HABITAT WITH INTERACTION OF TUMOR INFILTRATING LYMPHOCYTES (TILS) AND CANCER NUCLEI

(71) Applicant: Case Western Reserve University, Cleveland, OH (US)

(72) Inventors: Pranjal Vaidya, Cleveland, OH (US); Kaustav Bera, Cleveland, OH (US); Anant Madabhushi, Shaker Heights, OH (US)

(21) Appl. No.: 17/065,767

(22) Filed: Oct. 8, 2020

Related U.S. Application Data
(60) Provisional application No. 62/912,899, filed on Oct. 9, 2019.

(51) Int. Cl. G16H 50/20; G16B 40/30; G06T 7/11; G06T 7/00; G06K 9/46; G06K 9/62; G06N 3/04; G06N 3/08; G16H 30/20; G16H 30/40; G16H 50/30; G16H 50/50; G16H 70/60; G16H 50/70

(52) U.S. Cl. CPC G16H 50/20; G06T 2207/20084; G06T 7/11; G06T 7/0012; G06K 9/46; G06K 9/6256; G06N 3/04; G06N 3/088; G16H 30/20; G16H 30/40; G16H 50/30; G16H 50/50; G16H 70/60; G16H 50/70; G06T 2207/30061; G06T 2207/30096; G06T 2207/20081; G16B 40/30

(57) ABSTRACT

Embodiments discussed herein facilitate training and/or employing a machine learning model trained on radiomic features, quantitative histomorphometric features, and molecular expression to generate prognoses for treatment of tumors. One example embodiment can access a medical imaging scan of a tumor; segment a peri-tumoral region around the tumor; extract one or more radiomic features from the one or more of the tumor or the peri-tumoral region; provide the one or more radiomic features to a machine learning model trained based on the one or more radiomic features of a training set, one or more quantitative histomorphometric (QH) features of the training set, and a molecular expression of the training set; and receive a prognosis associated with the tumor from the machine learning model.

### CROSS REFERENCE TO RELATED APPLICATIONS

This application claims the benefit of U.S. Provisional Patent Application No. 62/912,899 filed Oct. 9, 2019, entitled “CT-DERIVED PROGNOSTIC RADIOMICS PHENOTYPE OF TUMOR HABITAT IS CLOSELY ASSOCIATED WITH INTERACTION OF TUMOR INFILTRATING LYMPHOCYTES (TILS) AND CANCER NUCLEI ON H&E TISSUE, AS WELL AS PD-L1 EXPRESSION IN NSCLC”, the contents of which are herein incorporated by reference in their entirety.

### BACKGROUND

Lung cancer is one of the most significant cause of cancer related deaths in both men as well as women. Annually, there are approximately 228,820 new lung cancer cases and 135,720 estimated deaths in the United States alone. Broadly lung cancer can be divided into small cell and non-small cell lung cancer (NSCLC) where NSCLC accounts for almost 85% of total cases. Early stage accounts for stage IA to IIB diseases and significant proportion of these patients have recurrent disease even after curative resection.

### BRIEF DESCRIPTION OF THE DRAWINGS

The accompanying drawings, which are incorporated in and constitute a part of the specification, illustrate various example operations, apparatus, methods, and other example embodiments of various aspects discussed herein. It will be appreciated that the illustrated element boundaries (e.g., boxes, groups of boxes, or other shapes) in the figures represent one example of the boundaries. One of ordinary skill in the art will appreciate that, in some examples, one element can be designed as multiple elements or that multiple elements can be designed as one element. In some examples, an element shown as an internal component of another element may be implemented as an external component and vice versa. Furthermore, elements may not be drawn to scale.

FIG. 1 illustrates a flow diagram of an example method/set of operations that can be performed by one or more processors to predict a prognosis for a potential treatment to a tumor based on a machine learning model trained on radiomic features, quantitative histomorphometric features, and molecular expression, according to various embodiments discussed herein.

FIG. 2 illustrates a flow diagram of an example method/set of operations that can be performed by one or more processors to train a machine learning model based on radiomic features, quantitative histomorphometric features, and molecular expression to predict a prognosis for a potential treatment to a tumor, according to various embodiments discussed herein.

FIG. 3 illustrates a diagram of an example apparatus that can facilitate training and/or employing a machine learning model to determine a prognosis (e.g., disease-free survival, etc.) based on a combination of two or more of radiomic features, quantitative histomorphometric (QH) features, and/or molecular phenotype, according to various embodiments discussed herein.

### DETAILED DESCRIPTION

Various embodiments discussed herein can Embodiments discussed herein facilitate training and/or employing a machine learning model trained on radiomic features, quantitative histomorphometric features, and molecular expression to generate prognoses for treatment of tumors. Embodiments can build and/or employ radio-histo-molecular phenotypes of tumor habitats stratified according to risk of recurrence, which can facilitate prediction of prognoses.

Some portions of the detailed descriptions that follow are presented in terms of algorithms and symbolic representations of operations on data bits within a memory. These algorithmic descriptions and representations are used by those skilled in the art to convey the substance of their work to others. An algorithm, here and generally, is conceived to be a sequence of operations that produce a result. The operations may include physical manipulations of physical quantities. Usually, though not necessarily, the physical quantities take the form of electrical or magnetic signals capable of being stored, transferred, combined, compared, and otherwise manipulated in a logic or circuit, and so on. The physical manipulations create a concrete, tangible, useful, real-world result.

It has proven convenient at times, principally for reasons of common usage, to refer to these signals as bits, values, elements, symbols, characters, terms, numbers, and so on. It should be borne in mind, however, that these and similar terms are to be associated with the appropriate physical quantities and are merely convenient labels applied to these quantities. Unless specifically stated otherwise, it is appreciated that throughout the description, terms including processing, computing, calculating, determining, and so on, refer to actions and processes of a computer system, logic, circuit, processor, or similar electronic device that manipulates and transforms data represented as physical (electronic) quantities.

Example methods and operations may be better appreciated with reference to flow diagrams. While for purposes of simplicity of explanation, the illustrated methodologies are shown and described as a series of blocks, it is to be appreciated that the methodologies are not limited by the order of the blocks, as some blocks can occur in different orders and/or concurrently with other blocks from that shown and described. Moreover, less than all the illustrated blocks may be required to implement an example methodology. Blocks may be combined or separated into multiple components. Furthermore, additional and/or alternative methodologies can employ additional, not illustrated blocks.

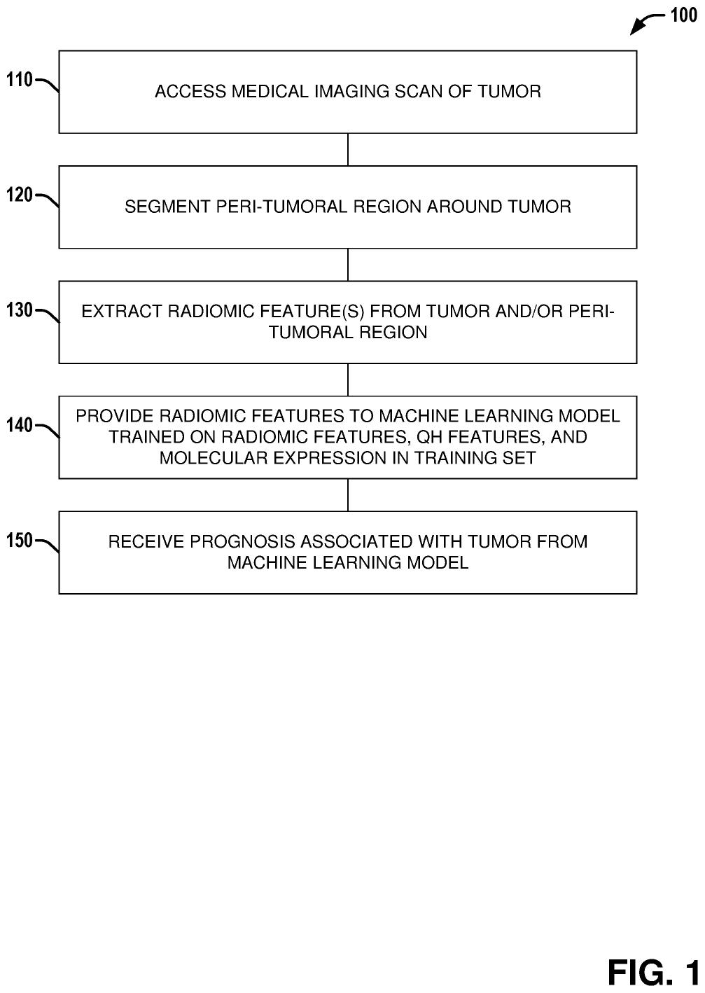

Referring to FIG. 1, illustrated is a flow diagram of an example method/set of operations 100 that can be performed by one or more processors to predict a prognosis for a potential treatment to a tumor based on a machine learning model trained on radiomic features, quantitative histomorphometric features, and molecular expression, according to various embodiments discussed herein. Processor(s) can include any combination of general-purpose processors and dedicated processors (e.g., graphics processors, application processors, etc.). The one or more processors can be coupled with and/or can include memory or storage and can be configured to execute instructions stored in the memory or storage to enable various apparatus, applications, or operating systems to perform the operations. The memory or storage devices may include main memory, disk storage, or any suitable combination thereof. The memory or storage devices can comprise—but is not limited to—any type of volatile or non-volatile memory such as dynamic random access memory (DRAM), static random-access memory (SRAM), erasable programmable read-only memory (EPROM), electrically erasable programmable read-only memory (EEPROM), Flash memory, or solid-state storage.

The set of operations 100 can comprise, at 110, accessing a medical imaging scan (e.g., MRI (contrast MRI, etc.), CT, etc.) of a tumor (e.g., segmented via expert annotation, computer segmentation (e.g., via deep learning, etc.), etc.). In various embodiments and in the example use case discussed below, the medical imaging scan can be obtained via a system and/or apparatus implementing the set of operations 100, or can be obtained from a separate medical imaging system (e.g., a MRI system/apparatus, a CT system/apparatus, etc.). Additionally, the medical imaging scan can be accessed contemporaneously with or at any point prior to performing the set of operations 100.

The set of operations 100 can further comprise, at 120, segmenting a peri-tumoral region around the tumor.

The set of operations 100 can further comprise, at 130, extracting one or more radiomic features from the one or more of the tumor or the peri-tumoral region.

The set of operations 100 can further comprise, at 140, providing the one or more radiomic features to a machine learning model trained based on the one or more radiomic features of a training set, one or more quantitative histomorphometric (QH) features of the training set, and a molecular expression of the training set (e.g., via unsupervised clustering on the radiomic features, followed by correlation with QH and molecular expression data).

The set of operations 100 can further comprise, at 150, receiving a prognosis associated with the tumor from the machine learning model.

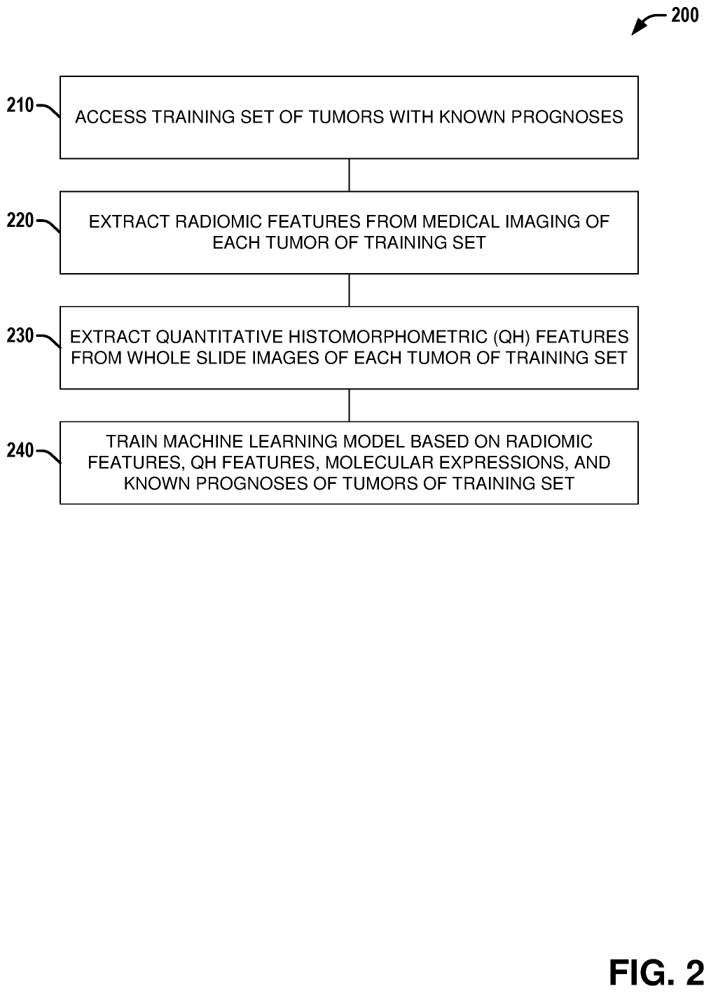

Referring to FIG. 2, illustrated is a flow diagram of an example method/set of operations 200 that can be performed by one or more processors to train a machine learning model based on radiomic features, quantitative histomorphometric features, and molecular expression to predict a prognosis for a potential treatment to a tumor, according to various embodiments discussed herein.

The set of operations 200 can comprise, at 210, accessing a training set a training set, wherein the training set comprises, for each tumor of a plurality of tumors: a medical imaging scan of that tumor, a whole slide image (WSI) of that tumor, a tissue-derived molecular expression for that tumor, and a known prognosis for that tumor. In various embodiments and in the example use case discussed below, the training set of medical imaging scans can be obtained via a system and/or apparatus implementing the set of operations 200, or can be obtained from a separate medical imaging system. Additionally, the training set can be accessed contemporaneously with or at any point prior to performing the set of operations 200.

The set of operations 200 can further comprise, at 220, for each tumor of the training set, extracting one or more radiomic features for that tumor from one of an intra-tumoral region of the medical imaging scan of that tumor or a peri-tumoral region around the intra-tumoral region.

The set of operations 200 can further comprise, at 230, for each tumor of the training set, extracting one or more quantitative histomorphometric (QH) features for that tumor from the WSI of that tumor.

The set of operations 200 can further comprise, at 240, for each tumor of the training set, training a machine learning model based on the one or more radiomic features for that tumor, the one or more QH features for that tumor, the tissue-derived molecular expression for that tumor, and the known prognosis for that tumor.

Additional aspects and embodiments are discussed below in connection with the following example use case.

### Example Use Case: CT-Derived Prognostic Radiomics Phenotype of Tumor Habitat is Closely Associated with Interaction of Tumor Infiltrating Lymphocytes (TILs) and Cancer Nuclei on H&E Tissue, as well as PD-L1 Expression In NSCLC

The following discussion provides example embodiments in connection with an example use case involving training, validation, and testing of models to generate a prognosis (disease free survival) for early stage non-small cell lung cancer (ES-NSCLC) based on a machine learning model trained to determine prognoses based on radio-histo-molecular tumor phenotypes.

Purpose: While radiomic analysis of lung nodules to predict outcome has been increasingly prevalent, the underlying tumor morphology that these features highlight is often not understood or explored. In the multi-modality analysis of the example use case, unique radiomic-histologic-molecular phenotypes for early stage non-small cell lung cancer (ES-NSCLC) patients were discovered which could successfully stratify patients based on their disease-free survival (DFS).

Materials & Methods: After retrospective chart review, a radiomic model was trained to predict the risk of recurrence following surgery for 316 ES-NSCLC patients using 124 radiomic textural features from the Gabor, Laws, Laplace, Haralick and Collage feature families extracted from a 0-3 mm annular ring immediately adjacent to the nodule (e.g., Peritumoral (PT) features extracted from a PT region). The radiomics model had an AUC (Area Under (ROC (Receiver Operating Characteristic)) Curve) of 0.78 (p<0.01) in predicting recurrence. Among 70 patients in this cohort, there was available tissue-derived PD-L1 expression, as well as H&E stained Whole slide images (WSIs). In order to build the radiomic-histologic-molecular phenotype of the tumor habitat, 242 Quantitative Histomorphometric (QH) features related to the nuclear shape, texture, orientation, spatial architecture of TILs and features quantifying TIL-cancer nuclei interaction were also extracted. Unsupervised clustering of the top 20 most discriminative features from 0-3 mm outside the tumor was done, and correlations of the clusters were calculated for QH and PDL-1 expression.

Results: Two significant clusters corresponding to high-risk and low-risk patients based on their risk of recurrence were obtained. The two clusters had significant disease-free survival (DFS) differences based on Kaplan-Meier analysis. (p<0.001). The two clusters were also correlated with nuclear morphology features (p<0.01) and spatial architecture of TIL patterns (p<0.01) as well as PD-L1 expression. It was found that the high-risk cluster had increased PD-L1 expression and increased intensity of the QH features.

Conclusion: The example use case built a radio-histo-molecular phenotype of the tumor habitat stratified according to the risk of recurrence in ES-NSCLC. It was found that these radiomic tumor habitat features were strongly correlated with TIL-cancer nuclei interaction and PD-L1 expression.

Clinical Relevance: The prognostic usefulness of radiomics of the tumor habitat can be complemented by understanding the underlying morphology in the tissue patterns which lead to the expression of these features, as shown in the example use case.

### ADDITIONAL EMBODIMENTS

In various example embodiments, method(s) discussed herein can be implemented as computer executable instructions. Thus, in various embodiments, a computer-readable storage device can store computer executable instructions that, when executed by a machine (e.g., computer, processor), cause the machine to perform methods or operations described or claimed herein including operation(s) described in connection with methods 100, 200, or any other methods or operations described herein. While executable instructions associated with the listed methods are described as being stored on a computer-readable storage device, it is to be appreciated that executable instructions associated with other example methods or operations described or claimed herein can also be stored on a computer-readable storage device. In different embodiments, the example methods or operations described herein can be triggered in different ways. In one embodiment, a method or operation can be triggered manually by a user. In another example, a method or operation can be triggered automatically.

Embodiments discussed herein relate to training and/or employing machine learning models (e.g., unsupervised (e.g., clustering) or supervised (e.g., classifiers, etc.) models) to determine a prognosis (e.g., likelihood of disease-free survival) for a tumor based on a combination of radiomic features and deep learning, based at least in part on features of medical imaging scans (e.g., MRI, CT, etc.) that are not perceivable by the human eye, and involve computation that cannot be practically performed in the human mind. As one example, machine learning classifiers and/or deep learning models as described herein cannot be implemented in the human mind or with pencil and paper. Embodiments thus perform actions, steps, processes, or other actions that are not practically performed in the human mind, at least because they require a processor or circuitry to access digitized images stored in a computer memory and to extract or compute features that are based on the digitized images and not on properties of tissue or the images that are perceivable by the human eye. Embodiments described herein can use a combined order of specific rules, elements, operations, or components that render information into a specific format that can then be used and applied to create desired results more accurately, more consistently, and with greater reliability than existing approaches, thereby producing the technical effect of improving the performance of the machine, computer, or system with which embodiments are implemented.

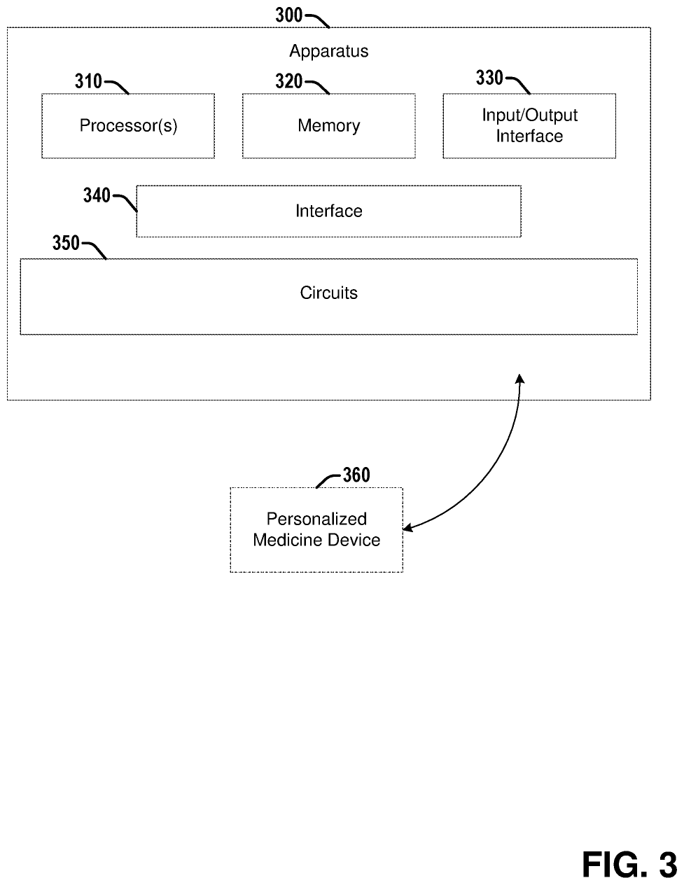

Referring to FIG. 3, illustrated is a diagram of an example apparatus 300 that can facilitate training and/or employing a machine learning model to determine a prognosis (e.g., disease-free survival, etc.) based on a combination of two or more of radiomic features, quantitative histomorphometric (QH) features, and/or molecular phenotype, according to various embodiments discussed herein. Apparatus 300 can be configured to perform various techniques discussed herein, for example, various operations discussed in connection with sets of operations 100 and/or 200. Apparatus 300 can comprise one or more processors 310 and memory 320. Processor(s) 310 can, in various embodiments, comprise circuitry such as, but not limited to, one or more single-core or multi-core processors. Processor(s) 310 can include any combination of general-purpose processors and dedicated processors (e.g., graphics processors, application processors, etc.). The processor(s) can be coupled with and/or can comprise memory (e.g., of memory 320) or storage and can be configured to execute instructions stored in the memory 320 or storage to enable various apparatus, applications, or operating systems to perform operations and/or methods discussed herein. Memory 320 can be configured to store medical imaging scan(s) (e.g., CT, MRI, stained (e.g., H&E) WSI or portion thereof, etc.) Each of the medical imaging scan(s) can comprise a plurality of pixels or voxels, each pixel or voxel having an associated intensity. Memory 320 can be further configured to store additional data involved in performing operations discussed herein, such as, radiomic and/or quantitative histomorphometric features, tissue-derived phenotype (e.g., PD-L1 expression, etc.), or other information employed in various methods (e.g., 100, 200, etc.) discussed in greater detail herein.

Apparatus 300 can also comprise an input/output (I/O) interface 330 (e.g., associated with one or more I/O devices), a set of circuits 350, and an interface 340 that connects the processor(s) 310, the memory 320, the I/O interface 330, and the set of circuits 350. I/O interface 330 can be configured to transfer data between memory 320, processor 310, circuits 350, and external devices, for example, a medical imaging device (e.g., CT system, MRI system, optical microscopy system, etc.), and/or one or more remote devices for receiving inputs and/or providing outputs to a clinician, patient, etc., such as optional personalized medicine device 360.

The processor(s) 310 and/or one or more circuits of the set of circuits 350 can perform one or more acts associated with a method or set of operations discussed herein, such as set of operations 100 and/or 200. In various embodiments, different acts (e.g., different operations of a set of operations) can be performed by the same or different processor(s) 310 and/or one or more circuits of the set of circuits 350.

Apparatus 300 can optionally further comprise personalized medicine device 360. Apparatus 300 can be configured to provide a prognosis (e.g., prediction related to disease-free survival, etc.) for a patient determined based at least in part on a combination of two or more of radiomic features, QH features, and/or molecular phenotype(s) and deep learning as discussed herein, and/or other data to personalized medicine device 360. Personalized medicine device 360 may be, for example, a computer assisted diagnosis (CADx) system or other type of personalized medicine device that can be used to facilitate monitoring and/or treatment of an associated medical condition. In some embodiments, processor(s) 310 and/or one or more circuits of the set of circuits 350 can be further configured to control personalized medicine device 360 to display the prognosis for a clinician or the patient or other data on a computer monitor, a smartphone display, a tablet display, or other displays.

Examples herein can include subject matter such as an apparatus, a medical imag system/apparatus, a personalized medicine system, a CADx system, a processor, a system, circuitry, a method, means for performing acts, steps, or blocks of the method, at least one machine-readable medium including executable instructions that, when performed by a machine (e.g., a processor with memory, an application-specific integrated circuit (ASIC), a field programmable gate array (FPGA), or the like) cause the machine to perform acts of the method or of an apparatus or system for generating system-independent quantitative perfusion measurements, according to embodiments and examples described.

Example 1 is a non-transitory computer-readable medium storing computer-executable instructions that, when executed, cause a processor to perform operations, comprising: accessing a medical imaging scan of a tumor; segmenting a peri-tumoral region around the tumor; extracting one or more radiomic features from the one or more of the tumor or the peri-tumoral region; providing the one or more radiomic features to a machine learning model trained based on the one or more radiomic features of a training set, one or more quantitative histomorphometric (QH) features of the training set, and a molecular expression of the training set; and receiving a prognosis associated with the tumor from the machine learning model.

Example 2 comprises the subject matter of any variation of any of example(s) 1, wherein the prognosis is one of disease-free survival (DFS) or non-DFS.

Example 3 comprises the subject matter of any variation of any of example(s) 1-2, wherein the one or more radiomic features comprise a first-order statistic of one or more of the following, extracted from the one of the medical imaging scan or the medical imaging scan after transformation with one of a filter or a wavelet decomposition: at least one Laws energy measure, at least one Gabor feature, at least one Haralick feature, at least one Laplace feature, at least one Co-occurrence of Local Anisotropic Gradient Orientations (CoLlAGe) feature, at least one Gray Level Size Zone Matrix, at least one Gray Level Run Length Matrix, at least one Neighboring Gray Tone Difference Matrix, at least one raw intensity value, at least one quantitative pharmacokinetic parameter, at least one semi-quantitative pharmacokinetic parameter, at least one Gray Level Dependence Matrix, at least one shape feature, or at least one feature from at least one pre-trained Convolutional Neural Network (CNN).

Example 4 comprises the subject matter of any variation of any of example(s) 3, wherein the first-order statistic is one of a mean, a median, a standard deviation, a skewness, a kurtosis, a range, a minimum, a maximum, a percentile, or histogram frequencies.

Example 5 comprises the subject matter of any variation of any of example(s) 1-4, wherein the one or more QH features comprise a feature associated with one or more of: a nuclear shape of the tumor, a nuclear texture of the tumor, a nuclear orientation of the tumor, a spatial architecture of tumor-infiltrating lymphocytes (TILs) of the tumor, or a TIL-nuclei interaction for the tumor.

Example 6 comprises the subject matter of any variation of any of example(s) 1-5, wherein the tumor is an early-stage non-small cell lung cancer (ES-NSCLC) tumor.

Example 7 comprises the subject matter of any variation of any of example(s) 1-6, wherein the machine learning model is an unsupervised clustering model.

Example 8 comprises the subject matter of any variation of any of example(s) 1-6, wherein the machine learning model is one of, or an ensemble of two or more of: a naïve Bayes classifier, a support vector machine (SVM) with a linear kernel, a SVM with a radial basis function (RBF) kernel, a linear discriminant analysis (LDA) classifier, a quadratic discriminant analysis (QDA) classifier, a logistic regression classifier, a decision tree, a random forest, a diagonal LDA, a diagonal QDA, a neural network, an AdaBoost algorithm, a LASSO, an elastic net, a Gaussian process classification, or a nearest neighbors classification.

Example 9 comprises the subject matter of any variation of any of example(s) 1-8, wherein the peri-tumoral region comprises an annular ring surrounding the tumor with a width between 2 mm and 4 mm.

Example 10 is an apparatus, comprising: a memory configured to store a medical imaging scan of a tumor; and one or more processors configured to: segment a peri-tumoral region around the tumor; extract one or more radiomic features from the one or more of the tumor or the peri-tumoral region; provide the one or more radiomic features to a machine learning model trained based on the one or more radiomic features of a training set, one or more quantitative histomorphometric (QH) features of the training set, and a molecular expression of the training set; and receive a prognosis associated with the tumor from the machine learning model.

Example 11 comprises the subject matter of any variation of any of example(s) 10, wherein the prognosis is one of disease-free survival (DFS) or non-DFS.

Example 12 comprises the subject matter of any variation of any of example(s) 10-11, wherein the one or more radiomic features comprise a first-order statistic of one or more of the following, extracted from the one of the medical imaging scan or the medical imaging scan after transformation with one of a filter or a wavelet decomposition: at least one Laws energy measure, at least one Gabor feature, at least one Haralick feature, at least one Laplace feature, at least one Co-occurrence of Local Anisotropic Gradient Orientations (CoLlAGe) feature, at least one Gray Level Size Zone Matrix, at least one Gray Level Run Length Matrix, at least one Neighboring Gray Tone Difference Matrix, at least one raw intensity value, at least one quantitative pharmacokinetic parameter, at least one semi-quantitative pharmacokinetic parameter, at least one Gray Level Dependence Matrix, at least one shape feature, or at least one feature from at least one pre-trained Convolutional Neural Network (CNN).

Example 13 comprises the subject matter of any variation of any of example(s) 12, wherein the first-order statistic is one of a mean, a median, a standard deviation, a skewness, a kurtosis, a range, a minimum, a maximum, a percentile, or histogram frequencies.

Example 14 comprises the subject matter of any variation of any of example(s) 10-13, wherein the one or more QH features comprise a feature associated with one or more of: a nuclear shape of the tumor, a nuclear texture of the tumor, a nuclear orientation of the tumor, a spatial architecture of tumor-infiltrating lymphocytes (TILs) of the tumor, or a TIL-nuclei interaction for the tumor.

Example 15 comprises the subject matter of any variation of any of example(s) 10-14, wherein the tumor is an early-stage non-small cell lung cancer (ES-NSCLC) tumor.

Example 16 comprises the subject matter of any variation of any of example(s) 10-15, wherein the machine learning model is an unsupervised clustering model.

Example 17 is a non-transitory computer-readable medium storing computer-executable instructions that, when executed, cause a processor to perform operations, comprising: accessing a training set, wherein the training set comprises, for each tumor of a plurality of tumors: a medical imaging scan of that tumor, a whole slide image (WSI) of that tumor, a tissue-derived molecular expression for that tumor, and a known prognosis for that tumor; for each tumor of the training set: extracting one or more radiomic features for that tumor from one of an intra-tumoral region of the medical imaging scan of that tumor or a peri-tumoral region around the intra-tumoral region; extracting one or more quantitative histomorphometric (QH) features for that tumor from the WSI of that tumor; and training a machine learning model based on the one or more radiomic features for that tumor, the one or more QH features for that tumor, the tissue-derived molecular expression for that tumor, and the known prognosis for that tumor.

Example 18 comprises the subject matter of any variation of any of example(s) 17, wherein, for each tumor of the training set, the one or more radiomic features for that tumor comprise a first-order statistic of one or more of the following, extracted from the one of the medical imaging scan or the medical imaging scan after transformation with one of a filter or a wavelet decomposition: at least one Laws energy measure, at least one Gabor feature, at least one Haralick feature, at least one Laplace feature, at least one Co-occurrence of Local Anisotropic Gradient Orientations (CoLlAGe) feature, at least one Gray Level Size Zone Matrix, at least one Gray Level Run Length Matrix, at least one Neighboring Gray Tone Difference Matrix, at least one raw intensity value, at least one quantitative pharmacokinetic parameter, at least one semi-quantitative pharmacokinetic parameter, at least one Gray Level Dependence Matrix, at least one shape feature, or at least one feature from at least one pre-trained Convolutional Neural Network (CNN).

Example 19 comprises the subject matter of any variation of any of example(s) 17-18, wherein, for each tumor of the training set, the one or more QH features for that tumor comprise a feature associated with one or more of: a nuclear shape of the tumor, a nuclear texture of the tumor, a nuclear orientation of the tumor, a spatial architecture of tumor-infiltrating lymphocytes (TILs) of the tumor, or a TIL-nuclei interaction for the tumor.

Example 20 comprises the subject matter of any variation of any of example(s) 17-19, wherein, for each tumor of the training set, the tissue-derived molecular expression for that tumor is a PD-L1 expression.

Example 21 comprises an apparatus comprising means for executing any of the described operations of examples 1-20.

Example 22 comprises a machine readable medium that stores instructions for execution by a processor to perform any of the described operations of examples 1-20.

Example 23 comprises an apparatus comprising: a memory; and one or more processors configured to: perform any of the described operations of examples 1-20.

References to “one embodiment”, “an embodiment”, “one example”, and “an example” indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

“Computer-readable storage device”, as used herein, refers to a device that stores instructions or data. “Computer-readable storage device” does not refer to propagated signals. A computer-readable storage device may take forms, including, but not limited to, non-volatile media, and volatile media. Non-volatile media may include, for example, optical disks, magnetic disks, tapes, and other media. Volatile media may include, for example, semiconductor memories, dynamic memory, and other media. Common forms of a computer-readable storage device may include, but are not limited to, a floppy disk, a flexible disk, a hard disk, a magnetic tape, other magnetic medium, an application specific integrated circuit (ASIC), a compact disk (CD), other optical medium, a random access memory (RAM), a read only memory (ROM), a memory chip or card, a memory stick, and other media from which a computer, a processor or other electronic device can read.

“Circuit”, as used herein, includes but is not limited to hardware, firmware, software in execution on a machine, or combinations of each to perform a function(s) or an action(s), or to cause a function or action from another logic, method, or system. A circuit may include a software controlled microprocessor, a discrete logic (e.g., ASIC), an analog circuit, a digital circuit, a programmed logic device, a memory device containing instructions, and other physical devices. A circuit may include one or more gates, combinations of gates, or other circuit components. Where multiple logical circuits are described, it may be possible to incorporate the multiple logical circuits into one physical circuit. Similarly, where a single logical circuit is described, it may be possible to distribute that single logical circuit between multiple physical circuits.

To the extent that the term “includes” or “including” is employed in the detailed description or the claims, it is intended to be inclusive in a manner similar to the term “comprising” as that term is interpreted when employed as a transitional word in a claim.

Throughout this specification and the claims that follow, unless the context requires otherwise, the words ‘comprise’ and ‘include’ and variations such as ‘comprising’ and ‘including’ will be understood to be terms of inclusion and not exclusion. For example, when such terms are used to refer to a stated integer or group of integers, such terms do not imply the exclusion of any other integer or group of integers.

To the extent that the term “or” is employed in the detailed description or claims (e.g., A or B) it is intended to mean “A or B or both”. When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995).

While example systems, methods, and other embodiments have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, and other embodiments described herein. Therefore, the invention is not limited to the specific details, the representative apparatus, and illustrative examples shown and described. Thus, this application is intended to embrace alterations, modifications, and variations that fall within the scope of the appended claims.

What is claimed is:

1. A non-transitory computer-readable medium storing computer-executable instructions that, when executed, cause a processor to perform operations, comprising:
accessing a medical imaging scan of a tumor;
segmenting a peri-tumoral region around the tumor;
extracting one or more radiomic features from the one or more of the tumor or the peri-tumoral region;
providing the one or more radiomic features to a machine learning model trained based on the one or more radiomic features of a training set, one or more quantitative histomorphometric (QH) features of the training set, and a molecular expression of the training set; and
receiving a prognosis associated with the tumor from the machine learning model.

2. The non-transitory computer-readable medium of claim 1, wherein the prognosis is one of disease-free survival (DFS) or non-DFS.

3. The non-transitory computer-readable medium of claim 1, wherein the one or more radiomic features comprise a first-order statistic of one or more of the following, extracted from the one of the medical imaging scan or the medical imaging scan after transformation with one of a filter or a wavelet decomposition: at least one Laws energy measure, at least one Gabor feature, at least one Haralick feature, at least one Laplace feature, at least one Co-occurrence of Local Anisotropic Gradient Orientations (CoLlAGe) feature, at least one Gray Level Size Zone Matrix, at least one Gray Level Run Length Matrix, at least one Neighboring Gray Tone Difference Matrix, at least one raw intensity value, at least one quantitative pharmacokinetic parameter, at least one semi-quantitative pharmacokinetic parameter, at least one Gray Level Dependence Matrix, at least one shape feature, or at least one feature from at least one pre-trained Convolutional Neural Network (CNN).

4. The non-transitory computer-readable medium of claim 3, wherein the first-order statistic is one of a mean, a median, a standard deviation, a skewness, a kurtosis, a range, a minimum, a maximum, a percentile, or histogram frequencies.

5. The non-transitory computer-readable medium of claim 1, wherein the one or more QH features comprise a feature associated with one or more of: a nuclear shape of the tumor, a nuclear texture of the tumor, a nuclear orientation of the tumor, a spatial architecture of tumor-infiltrating lymphocytes (TILs) of the tumor, or a TIL-nuclei interaction for the tumor.

6. The non-transitory computer-readable medium of claim 1, wherein the tumor is an early-stage non-small cell lung cancer (ES-NSCLC) tumor.

7. The non-transitory computer-readable medium of claim 1, wherein the machine learning model is an unsupervised clustering model.

8. The non-transitory computer-readable medium of claim 1, wherein the machine learning model is one of, or an ensemble of two or more of: a naïve Bayes classifier, a support vector machine (SVM) with a linear kernel, a SVM with a radial basis function (RBF) kernel, a linear discriminant analysis (LDA) classifier, a quadratic discriminant analysis (QDA) classifier, a logistic regression classifier, a decision tree, a random forest, a diagonal LDA, a diagonal QDA, a neural network, an AdaBoost algorithm, a LASSO, an elastic net, a Gaussian process classification, or a nearest neighbors classification.

9. The non-transitory computer-readable medium of claim 1, wherein the peri-tumoral region comprises an annular ring surrounding the tumor with a width between 2 mm and 4 mm.

10. An apparatus, comprising:
a memory configured to store a medical imaging scan of a tumor; and
one or more processors configured to:
segment a peri-tumoral region around the tumor;
extract one or more radiomic features from the one or more of the tumor or the peri-tumoral region;
provide the one or more radiomic features to a machine learning model trained based on the one or more radiomic features of a training set, one or more quantitative histomorphometric (QH) features of the training set, and a molecular expression of the training set; and
receive a prognosis associated with the tumor from the machine learning model.

11. The apparatus of claim 10, wherein the prognosis is one of disease-free survival (DFS) or non-DFS.

12. The apparatus of claim 10, wherein the one or more radiomic features comprise a first-order statistic of one or more of the following, extracted from the one of the medical imaging scan or the medical imaging scan after transformation with one of a filter or a wavelet decomposition: at least one Laws energy measure, at least one Gabor feature, at least one Haralick feature, at least one Laplace feature, at least one Co-occurrence of Local Anisotropic Gradient Orientations (CoLlAGe) feature, at least one Gray Level Size Zone Matrix, at least one Gray Level Run Length Matrix, at least one Neighboring Gray Tone Difference Matrix, at least one raw intensity value, at least one quantitative pharmacokinetic parameter, at least one semi-quantitative pharmacokinetic parameter, at least one Gray Level Dependence Matrix, at least one shape feature, or at least one feature from at least one pre-trained Convolutional Neural Network (CNN).

13. The apparatus of claim 12, wherein the first-order statistic is one of a mean, a median, a standard deviation, a skewness, a kurtosis, a range, a minimum, a maximum, a percentile, or histogram frequencies.

14. The apparatus of claim 10, wherein the one or more QH features comprise a feature associated with one or more of: a nuclear shape of the tumor, a nuclear texture of the tumor, a nuclear orientation of the tumor, a spatial architecture of tumor-infiltrating lymphocytes (TILs) of the tumor, or a TIL-nuclei interaction for the tumor.

15. The apparatus of claim 10, wherein the tumor is an early-stage non-small cell lung cancer (ES-NSCLC) tumor.

16. The apparatus of claim 10, wherein the machine learning model is an unsupervised clustering model.

17. A non-transitory computer-readable medium storing computer-executable instructions that, when executed, cause a processor to perform operations, comprising:
accessing a training set, wherein the training set comprises, for each tumor of a plurality of tumors: a medical imaging scan of that tumor, a whole slide image (WSI) of that tumor, a tissue-derived molecular expression for that tumor, and a known prognosis for that tumor;
for each tumor of the training set:
extracting one or more radiomic features for that tumor from one of an intra-tumoral region of the medical imaging scan of that tumor or a peri-tumoral region around the intra-tumoral region;
extracting one or more quantitative histomorphometric (QH) features for that tumor from the WSI of that tumor; and
training a machine learning model based on the one or more radiomic features for that tumor, the one or more QH features for that tumor, the tissue-derived molecular expression for that tumor, and the known prognosis for that tumor.

18. The non-transitory computer-readable medium of claim 17, wherein, for each tumor of the training set, the one or more radiomic features for that tumor comprise a first-order statistic of one or more of the following, extracted from the one of the medical imaging scan or the medical imaging scan after transformation with one of a filter or a wavelet decomposition: at least one Laws energy measure, at least one Gabor feature, at least one Haralick feature, at least one Laplace feature, at least one Co-occurrence of Local Anisotropic Gradient Orientations (CoLlAGe) feature, at least one Gray Level Size Zone Matrix, at least one Gray Level Run Length Matrix, at least one Neighboring Gray Tone Difference Matrix, at least one raw intensity value, at least one quantitative pharmacokinetic parameter, at least one semi-quantitative pharmacokinetic parameter, at least one Gray Level Dependence Matrix, at least one shape feature, or at least one feature from at least one pre-trained Convolutional Neural Network (CNN).

19. The non-transitory computer-readable medium of claim 17, wherein, for each tumor of the training set, the one or more QH features for that tumor comprise a feature associated with one or more of: a nuclear shape of the tumor, a nuclear texture of the tumor, a nuclear orientation of the tumor, a spatial architecture of tumor-infiltrating lymphocytes (TILs) of the tumor, or a TIL-nuclei interaction for the tumor.

20. The non-transitory computer-readable medium of claim 17, wherein, for each tumor of the training set, the tissue-derived molecular expression for that tumor is a PD-L1 expression.
